(19) United States
(12) Patent Application Publication
(10) Pub. No.: US 2004/0241677 A1
(43) Pub. Date: Dec. 2, 2004

(54) Techniques for automated diagnosis of cell-borne anomalies with digital optical microscope

(76) Inventors: Jeffrey S Lin, Silver Spring, MD (US); Andrew B Feldman, Columbia, MD (US); Plamen A Demirev, Ellicott City, MD (US); Peter F Scholl, Silver Spring, MD (US); Sean P Murphy, Silver Spring, MD (US)

Correspondence Address: Francis A Cooch, The John Hopkins University, Applied Physics Laboratory, 111000 Johns Hopkins Road, Laurel, MD, 20723-6099, US

(21) Appl. No.: 10/477,648

(22) PCT Filed: May 29, 2003
(86) PCT No.: PCT/US03/16692

(51) Int. Cl. C12Q 1/68; C12Q 1/44; G06F 19/00; G01N 33/48; G01N 33/50

(52) U.S. Cl. 435/6; 435/40.5; 702/19

(57) ABSTRACT

Techniques for automatically analyzing a biological sample with a microscope include obtaining a first digital image of a first field of view of the biological sample. Cell data and anomalous data are automatically determined. Cell data indicates an area co-located in the first digital image with a cell set of one or more cells of a particular type. Anomalous data indicates an area co-located in the first digital image with an anomalous set of zero or more particular objects that are anomalous to normal cells of the particular type. The cell data and the anomalous data are automatically combined to determine the particular objects inside the cell set in the first digital image. An analytical result for the biological sample is generated based on the particular objects inside the cell set. These techniques allow the automated classification and quantification of malaria in microscope views of blood smears, among other diseases.

### CROSS-REFERENCE TO RELATED APPLICATIONS

This application claims benefit of Provisional Appln. 60/384,323, filed May 30, 2002, the entire contents of which are hereby incorporated by reference as if fully set forth herein, under 35 U.S.C. §119(e).

### BACKGROUND OF THE INVENTION

1. Field of the Invention

The present invention relates to automated analysis of microscope data; and, in particular, to the automated analysis of anomalous bodies, such as parasites, inside host cells in microscope data.

2. Description of the Related Art

Malaria is a disease affecting man and other animals, caused by different species of the parasite Plasmodia that are transmitted by mosquito bites. During a complex life cycle involving insect and animal hosts, the malaria parasite invades and destroys red blood cells (erythrocytes). Despite intense efforts to combat it, malaria still afflicts more than 500 million people. Each year, malaria infections kill between one and two million people in 103 countries, predominantly children. The emergence of drug-resistant strains and slow progress in developing an effective vaccine has compounded efforts to control the spread of the disease. Malaria in humans is caused by four different species of protozoans in the genus Plasmodium: P. falciparum; P. vivax; P. malariae and P. ovale, with P. falciparum being the most lethal.

The Plasmodium life cycle proceeds through several asexual and sexual stages. Plasmodium sporozoites, transmitted by female Anopheles mosquitoes, are injected into the blood of an animal host together with mosquito saliva. After initial proliferation in the liver, parasites in the merozoite stage are released back into the blood stream. A single merozoite then invades a red blood cell and matures by forming a ring-shaped cell. In about 24 hours the matured parasite enters the trophozoite stage, during which most of the red blood cell cytoplasm, including hemoglobin, is catabolized. Through the final, schizont, stage in the red blood cell, the parasite undergoes several divisions to produce up to thirty-two new merozoites that burst the host red blood cell and invade new red blood cells.

Rapid, sensitive and reliable methods for malaria detection are a factor that determines the ultimate success in controlling, restricting and eradicating this disease. Moreover, accurate parasitemia quantification, the number of parasites per unit of blood, is indispensable in malaria treatment, in screening new drug and candidate vaccine efficacy, and in identifying the emergence of drug-resistant parasite strains. While optical microscopy of Giemsa-stained blood smears is still considered the “gold standard” for malaria detection, it is a time consuming process requiring about one half hour per diagnosis using experienced technicians.

In the optical microscopy “gold-standard,” a combination of thick-smear and thin-smear slides of blood samples from an individual are used, typically, to detect the infection, to enumerate the parasites, and then to classify the infectious agent. For malaria, a magnification of 1000×(total) is typically used to identify the parasite's species and stage of development based on the parasite shape and location inside a red blood cell. At such magnification, very few parasites are visible in a microscope's field of view on a thin-smear slide, especially at early stages of the infection. Therefore the human technician manually searches through a number of fields of view until a classification of species and stage can be made and sufficient counts of red blood cell and parasites are accumulated to enumerate the parasitemia level or rule out an infection. The level of parasitemia is estimated either from the number of infected red blood cell per unit of volume of blood, or from direct counts of the observed parasites per unit volume of blood. The unit volume can be estimated from the total number of red blood cells, or estimated from a count of the white blood cells, which are fewer in number and easier to count, and the ratio of red blood cells to white bloods cells in normal blood.

Some of these approaches introduce errors. For example, an infected individual may have a different ratio of red blood cell per unit volume or a different ratio of red blood cell to white blood cells, or both, so that the unit volume estimate, and the dependent parasitemia level, are imprecise.

The process is manual and therefore subjective to some degree. The parasitemia levels determined for the same sample can vary between different passes by the same technician, not only due to errors in the method but in the state of concentration of the technician, and can vary even more by different passes by different technicians of different skill levels. For example, a skilled and careful and rested technician can detect Plasmodia parasites at parasitemia levels as low as about 5 to 10 parasites per microliter (μl, 1 μl=10⁻⁶ liters). A more practical detection limit by an average technician is about 50 to 100 parasites per μl (p/μl).

The process is time consuming. The time required for preparation and inspection of a single slide is on the order of 60 minutes according to a recent article (New Perspectives: Malaria Diagnosis, World Health Organization, Geneva, 2000). While detection of parasites for the case of a high parasitemia level (5,000 to 10,000 p/μl) typically consumes much less than 60 minutes, the classification of a single sample as “negative for the presence of parasites” can involve examining 100 to 300 microscope fields of view and consume a full 60 minutes. The correct classification of a sample as “negative” is extremely important, since a missed detection, i.e., a “false negative,” can have lethal consequences, particularly for an infection of P. falciparum in a child.

Furthermore, the present techniques are costly, requiring many person-hours to screen the samples from even a small population of exposed individuals.

Another disadvantage of the present techniques is that the slides examined and characterized locally by a technician are not available to a remote expert who may be better able to determine the extent of an outbreak of the disease. One approach to making such slides available is to package and ship the slides to that expert. Disadvantages of this approach are that the slides are unavailable during shipment, and are not made available to multiple experts at multiple remote sites. In another approach, the technician makes photographs of the fields of view on each slide and copies or scans the photographs for dissemination to remote experts. A disadvantage of this approach is that it involves a separate step to photograph each field of view, which step adds to the time to analyze each slide, and therefore further slows the analyses and increases the costs of those analyses.

In addition to malaria, other diseases may be characterized by the infestation of parasites or by the accumulation or generation of anomalous features caused by smaller pathological agents in cells, such as Babesiosis. Such diseases can also be diagnosed by human examination of optical microscope slides for anomalies in cells. Such manual diagnosis methods often also suffer disadvantages from errors, subjectivity, time consumption, high costs or other deficiencies, or some combination of these.

Techniques to automate the examination of microscope slides with biological samples are known. For example, a recent patent, U.S. Ser. No. 6,005,964, by Reid et al., entitled “Automatic machine vision microscope slide inspection system and method” and issued Dec. 21, 1999 (hereinafter referenced as Reid), the entire contents of which are hereby incorporated by reference as if fully set forth herein, describes techniques to examine and analyze microscope slides under control of a computer system to automatically detect candidate structures that may indicate parasites outside animal cells that contaminate drinking water and to automatically classify the candidates as such parasites. However, these known techniques do not result in automated identification of anomalies, such as parasites of various stages within cells, or in automated quantification of parasitemia levels in blood for malaria.

Based on the foregoing, there is a clear need for techniques that automate the classification or quantification of anomalous bodies in cells, or both, by inspecting optical microscope slides, which do not suffer the errors, subjectivity, time consumption, high costs, limited availability, or other deficiencies of the prior art.

In particular, there is a clear need for techniques that automate the classification or quantification of parasite infestations in blood cells, or both, by inspecting optical microscope slides, which do not suffer from one or more deficiencies of the prior art.

In particular, furthermore, there is a clear need for techniques that automate the detection of malaria, the identification of malaria parasite stages in blood cells, or the quantification of the parasitemia level of malaria in an infected animal, or some combination of these, by inspecting optical microscope slides, which do not suffer from one or more deficiencies of the prior art.

### SUMMARY OF THE INVENTION

Techniques for automatically analyzing a biological sample with a microscope include obtaining a first digital image of a first field of view of the biological sample. Cell data and anomalous data are automatically determined. Cell data indicates an area co-located in the first digital image with a cell set of one or more cells of a particular type. Anomalous data indicates an area co-located in the first digital image with an anomalous set of zero or more particular objects that are anomalous to normal cells of the particular type. The cell data and the anomalous data are automatically combined to determine the particular objects inside the cell set in the first digital image. An analytical result for the biological sample is generated based on the particular objects inside the cell set.

These techniques allow the automated classification and quantification of malaria in microscope views of blood smears, among other diseases.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a grayscale version of a color digital image that illustrates a portion of a microscope field of view of a thin-smear blood sample;

FIG. 2 is a block diagram that illustrates a system for automatically collecting and analyzing digital images of microscope fields of view, according to an embodiment;

FIG. 3 is a flow diagram of a method for automatically collecting and analyzing digital images of microscope fields of view, according to an embodiment;

FIG. 4A is a grayscale version of a digital image that illustrates red blood cell data from the field of view of FIG. 1, according to an embodiment;

FIG. 4B is a grayscale version of a digital image that illustrates parasite data from the field of view of FIG. 1, according to an embodiment; and

FIG. 5 is a block diagram that illustrates a computer system upon which an embodiment of the invention may be implemented.

### DETAILED DESCRIPTION

A method and apparatus are described for the automated analysis of microscope slides for anomalous bodies inside cells. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Embodiments of the invention are described primarily in the context of transmission optical microscope views of thin blood smears for malaria parasite infestation of human red blood cells, but the invention is not limited to this context. For example, in other embodiments the techniques may be applied to malaria in animals. In other embodiments the techniques may be applied to views of biological samples generated by reflection or with non-visible optical wavelengths, such as infrared and ultraviolet wavelengths of the electromagnetic spectrum, either directly or through fluorescence. Other embodiments may be directed to other diseases that cause abnormal formations or biological entities to appear inside cells, such as babesiosis.

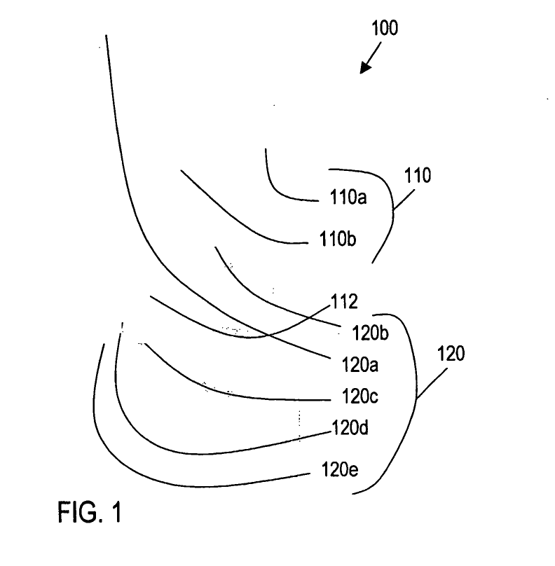

For purposes of illustration, an example field of view of an optical microscope is used to demonstrate the techniques. FIG. 1 is a grayscale version of a color digital image 100 that illustrates a portion of a microscope field of view of a thin-smear blood sample. The slide has been treated with a Giemsa stain to enhance the appearance of parasites.

The image 100 depicts normal red blood cells 110, such as normal red blood cells 110a, 110b, as light gray circular objects of a well-known uniform radius. In a color digital image, these may appear as red or blue in color. Some red blood cells appear as rings, e.g. red blood cell 110b, and others as more solid filled circles, e.g. 110a. These appearances are related to the depth of focus of the field of view of the microscope relative to the central portion of the red blood cell, which is thinner than the outer ring portion of the cell. As is apparent in FIG. 1, some red blood cells overlap others. Also, some red blood cells may be imaged in a side view, if the cell is oriented in a plane that is more perpendicular to the plane of the field of view. In addition, some red blood cells may be deformed as a result of the presence of infectious agent or other pathology. For example red blood cell 112 is deformed by a parasite inside the cell.

Also shown in FIG. 1 are dark bodies 120, including dark bodies 120a, 120b, 120c, 120d, 120e, that have taken up the Giemsa stain. In a color digital image these may appear as purple in color in addition to being dark. These dark bodies have different sizes and shapes than red blood cells. At least red blood cell 112 includes multiple dark bodies 120d, 120e within itself. The presence of stained dark bodies 120d, 120e within red blood cell 112 indicates a malaria infestation in this example blood sample.

1. Structural Overview

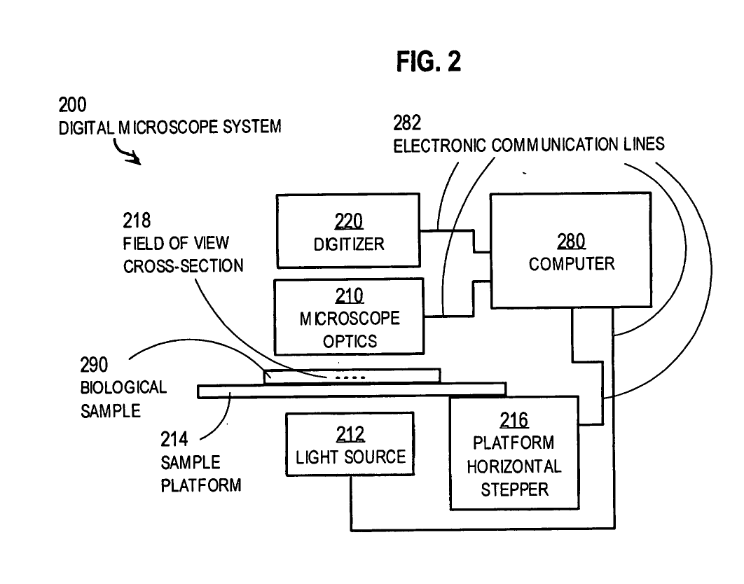

FIG. 2 is a block diagram that illustrates a system 200 for automatically collecting and analyzing digital images of microscope fields of view, according to an embodiment. In system 200, a computer 280 controls several elements of a digital microscope through electronic communication lines 282. The illustrated digital microscope includes a light source 212, a sample platform 214, a platform horizontal stepper 216, microscope optics 210 and image digitizer 220.

A biological sample 290 is placed on sample platform 214 for viewing by the digital microscope. If transmission microscopy is employed, then the platform 214 is transparent to light at the wavelengths of interest where the sample is placed on the platform.

The horizontal stepper 216 moves the platform 290 horizontally, for example, incrementally in each of two horizontal directions. For purposes of illustration, it is assumed that the two horizontal directions are parallel to the long dimension of the sample platform 214 and perpendicular to the page of FIG. 2. The horizontal stepper 216 may be manually controlled in some embodiments. In the illustrated embodiments, a process executing on computer 280 controls the horizontal stepper 216.

The light source 212 provides light to illuminate the biological sample for imaging by the microscope. Any light source known in the art may be used, including a source of non-visible infrared and ultraviolet wavelengths of the electromagnetic spectrum. The light source may be disposed in any position known in the art for illuminating a biological sample, including below the sample, as depicted, for transmission microscopy, or above the sample for reflection microscopy. The light source 212 may be controlled in some embodiments in terms of an on/off state, or intensity, or spectrum of wavelengths, or other property, or in some combination. The light source 212 may be manually controlled in some embodiments. In the illustrated embodiments, a process executing on computer 280 controls the light source 212.

The microscope optics 210 uses one or more lenses of one or more focal lengths to magnify a view of a portion of the biological sample. For example, a cross-section of a field of view 218 in the sample 290 is depicted in FIG. 2. This field of view is magnified and output as an optical image by the microscope optics. The degree of magnification is adjusted by control of one or more of the adjustable lenses. The depth of the field of view in the sample 290 is also adjusted by control of one or more adjustable lenses. The depth is perpendicular to the two horizontal directions. The horizontal position of the field of view 218 in the sample is controlled by the movement of the platform 214 by the horizontal stepper 216, as described above. The lenses of microscope optics 210 may be manually controlled in some embodiments. In the illustrated embodiments, a process executing on computer 280 controls the lenses of microscope optics 210.

The digitizer 220 produces a digital image of the optical image output by the microscope optics. A digital image is made up a finite number of picture elements (pixels). Any method known in the art may be used to produce the digital image. For example, a row of optical detectors may be scanned across the optical image to record light intensity. In some embodiments, color filters or other mechanisms are used to determine intensity as a function of two or more colors, called color planes. In some embodiments a color digital camera with a two dimensional array of detectors and three color planes is used. The digitizer 220 may be manually controlled in some embodiments. In the illustrated embodiments, a process executing on computer 280 controls the digitizer 220.

The digital image of the field of view 218 is sent as data to the computer 280 for further processing, as described below.

2. Method for Detecting Anomalies in Cells

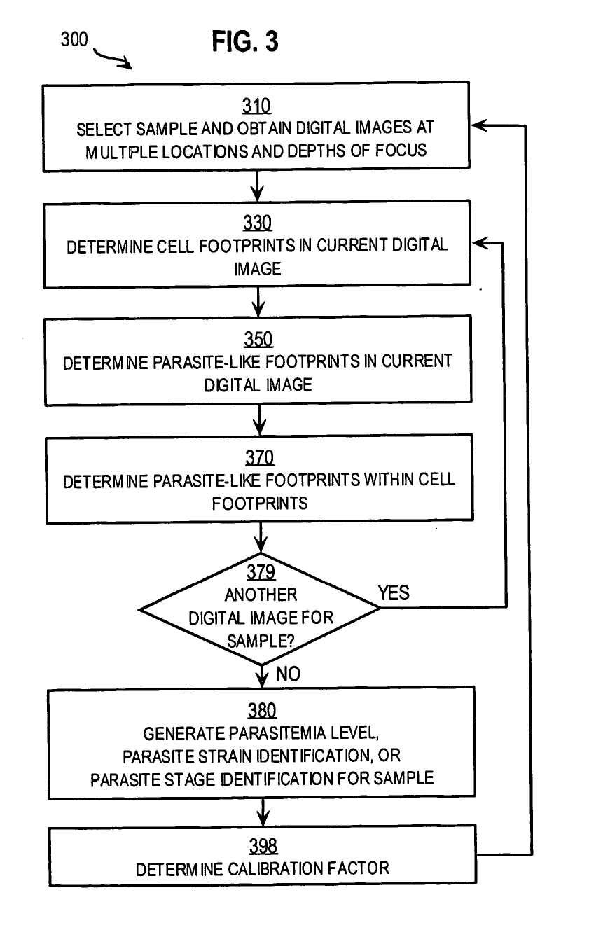

FIG. 3 is a flow diagram of a method 300 for automatically collecting and analyzing digital images of microscope fields of view, according to an embodiment.

In step 310, a sample of biological material is selected and one or more digital images of fields of views from one or more horizontal locations and depths of focus are obtained from the sample. In some embodiments, photographs are taken and digitized manually, or digital photographs are taken manually, and downloaded to computer 280. In the illustrated embodiment, the digital microscope of FIG. 2 is controlled by computer 280 to generate the digital images obtained in step 310.

For example, the stepper 216 is controlled to align a particular horizontal location of the sample along a viewing axis of the microscope optics, and the microscope optics are controlled to focus on a plane of the sample at a depth closest to the microscope optics. A digital camera serving as digitizer 220 is activated to capture a first digital image and the first image is downloaded to computer 280. In the illustrated embodiments, each digital image includes a set of pixels for the field of view in each of three color planes, one red (R), one green (G) one blue (B); such digital images are called RGB digital images. The microscope optics are again controlled to focus on a second field of view in the sample at a depth farther from the microscope optics. The digital camera is activated again to capture a second digital image and the second image is downloaded to computer 280. The process is repeated as often as desired to obtain a set of digital images for analysis.

In some embodiments, the difference in depth of focus between the first and second digital images is selected to resolve the features of interest. For example, the change in depth of focus is chosen to match half the shortest dimension of a parasite to ensure that no parasite is missed in the digital images taken at the current particular horizontal location. In one embodiment, the increment in depth of focus is 0.5 μm because a parasite is expected to be about 1 μm in size. Since a typical thin smear sample is about 5 μm thick, about 10 depths of focus are employed for each horizontal position.

In some embodiments, the changes in horizontal location, between one set of digital images at various depths of focus and a second set of digital images at various depths of focus, is also chosen to avoid missing any parasites. Thus the change in horizontal location in each of two horizontal directions is selected to match the size of the field of view in the direction moved. In some embodiments, only a statistically valid sub-sample of the biological sample is to be analyzed in order to achieve a result within certain bounds of statistical confidence. In these embodiments, the change in horizontal location is selected to sub-sample a percentage of the biological sample determined for those bounds on statistical confidence. For example, if only 100 horizontal locations are selected for the desired degree of statistical confidence when a complete examination of the biological sample would require 400 horizontal locations, then the horizontal location is selected to sub-sample 25% of the biological sample.

In some embodiments, step 310 includes storing the digital images in a database that can be accessed by remote experts, for example over a network or by automatically generation and sending digital images on some computer-readable medium, as described in more detail in a later section.

In step 330, cell data is determined in the current digital image. The current digital image is one image selected from the digital images obtained above, in step 310. In the illustrated embodiment, step 330 includes determining red-blood-cell footprints in the current digital image. Any method of determining a cell footprint may be used.

In an illustrated embodiment, a RGB digital image is separated into three separate digital images, one for each color plane, and one or more of the color planes are selected to determine the cell footprint of a red blood cell. For example, in one embodiment, the red plane is selected. In some embodiment, pixels from multiple planes are combined in a weighted average. In the weighted average, the weight can be zero for one or more color planes. In an illustrated embodiment, a pixel of the selected plane (or weighted average) is considered within a red blood cell if its value is above a particular threshold value (called herein a red-blood-cell threshold). An initial red-blood-cell footprint for the image is made up of all the pixels above the threshold.

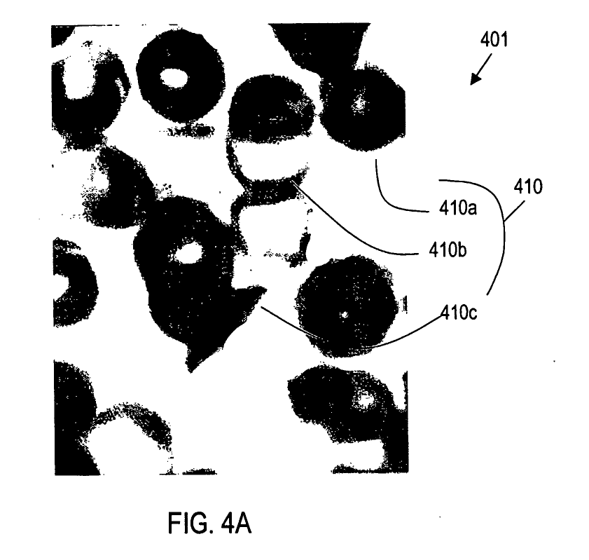

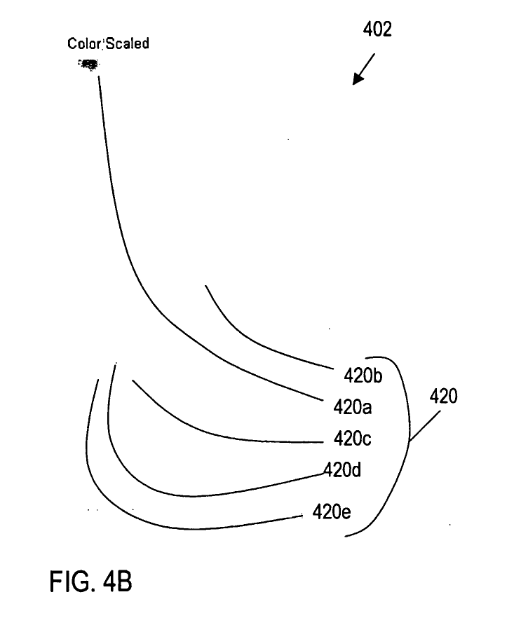

FIG. 4A is a grayscale version of a digital image that illustrates red blood cell data from the field of view of FIG. 1. All pixels below a red-blood-cell threshold are white in this image. The dark pixels make up an initial red-blood-cell footprint. The red-blood-cell footprint includes red blood cells 410, such as 410a, 410b, 410c. Essentially all pixels in normal red blood cell 410a, and in sideways, abnormal red blood cell 410c, are included in this initial footprint. Many pixels inside red blood cell 410b, however, are not included in this initial red-blood-cell footprint. This footprint may be augmented by pixels above the threshold in another digital image at another depth of focus, or by other methods, such as described below.

In some embodiments, other well-known image processing techniques are used instead of, or in addition to, determining pixels above a particular threshold. For example, in some embodiments, gradations in the illumination field are measured and subtracted from the selected color plane or weighted average before applying the threshold. In some embodiments, an edge detector is used to find an edge between pixels that are included in the red-blood-cell footprint and those that are not. These edges, combined with morphology criteria define the interior of red blood cells.

In some digital images, the set of pixels indicating red blood cells are not smooth on the scale of the well-known red blood cell radius and this may cause application of a threshold to leave pixels outside the cell that ought to be included inside the red blood cells. Well known image-processing techniques can be employed to include these excluded pixels. One well-known technique is morphological binary closing. In this technique, objects are dilated by adding pixels that are up to a first parametric distance from pixels inside the cell. After dilation, there is an erosion that deletes pixels that are up to a second, sometimes different parametric distance from those pixels still outside the cell. The size of the parametric distance, or distances, is selected based on the properties of the expected cell footprints. For example, at 1000 times magnification and digital images with a known number of pixels per unit length in each dimension, one can calculate the number of pixels across the radius of a typical red blood cell, and the parameter is chosen as some fraction of the typical radius. The net effect of this technique is to smooth the outer boundaries of the cells, to fill narrow inlets and small cavities, and to connect narrowly separated objects. For example, the initial red-blood-cell footprint determined in FIG. 4A can be expanded by this technique to include more or all the pixels inside red blood cell 410b.

In some embodiments, step 330 includes counting red blood cells in the digital image. Any method known in the art may be used to count the red blood cells in the red-blood-cell footprint. In one method, the area of the red-blood-cell footprint is divided by the area of a circle that has a radius equal to an appropriate mean red blood cell radius. Alternatively, one can apply morphological erosion techniques to reduce the cell footprints to a collection of centers that can be enumerated. In some embodiments, step 330 includes counting white blood cells.

In some embodiments, step 330 includes storing the cell data in the database that can be accessed by remote experts, in association with the corresponding digital image.

In step 350, anomaly data is determined in the current digital image. In the illustrated embodiment, step 350 includes determining parasite footprints in the current digital image. Any method of determining an anomaly footprint may be used, such as applying thresholds and edge detection, as described above for determining the red blood cell data during step 330.

For example, the parasites are stained purple with the Giemsa stain. Color filtering can be used before applying a threshold to pick Giemsa-stained bodies out of the current digital image. With an RGB image, each pixel is a 3 element vector describing the intensity of the red, green, and blue color at that location in the image. Color filtering involves the selection of a three dimensional subregion within the space of all possible RGB values determined by the minimum and maximum intensities for each of the colors in the image being analyzed. Pixels whose RGB values fall outside the boundaries of this selected subregion are eliminated (mapped to white). The desired boundaries of this color filter can be hardcoded into the algorithm via prior image analysis or can be determined at run-time via automated segmentation techniques. FIG. 4B is a grayscale version of a digital image that illustrates parasite data from the field of view of FIG. 1, according to an embodiment using color filtering. In this example, only pixels with RGB combinations that qualify as purple are retained. The normal red blood cells are gone from this image. Only the parasite candidates 420a, 420b, 420c, 420d, 420e remain, corresponding to dark bodies 120a, 120b, 120c, 120d, 120e of FIG. 1, respectively.

In some embodiments, step 350 includes storing the anomaly data in the database that can be accessed by remote experts in association with the corresponding digital image.

In step 370, the anomalous bodies inside the cells are determined by combining the cell data and the anomaly data. In the illustrated embodiment, step 370 includes determining the parasite footprints in the current digital image that are inside the cell footprint. Any method of determining whether one footprint is inside another footprint may be used. In the illustrated embodiment, the pixels in the red-blood-cell footprint are given the value 1 and the pixels outside are given the value zero. Similarly, the pixels in the stained dark body footprint are given the value 1 and the pixels outside the stained dark body footprint are given the value zero. A pixel-by pixel logical AND is performed that gives the value 1 if and only if the values of the two pixels from the same location in the red-blood-cell footprint and stained dark body footprint are both 1, and gives a value zero otherwise. The result of the pixel-by-pixel logical and is a footprint of stained parasites inside red blood cells. For example, pixels in Giemsa-stained bodies 420d, 420e are also in the red-blood-cell footprint, so these bodies are retained as a result of combining the footprints. Giesma-stained bodies 420a, 420b, 420c are not inside red blood cells and do not qualify for further analysis. These bodies may represent parasites in the merozoite stage that are found for a short period outside red blood cells, after an infested cell has burst.

In some embodiments, step 370 includes counting the anomalous bodies inside the cells in the current digital image. Any method known in the art can be used to count the dark bodies inside the cells. In some embodiments, before counting, the parasite footprints are completed, in case they are disjoint or ragged, using binary morphological closing, as described above. In some embodiments, the count is determined by dividing the number of pixels in the anomalous bodies inside the cells by the number of pixels in the average size anomaly, such as a known parasite size. In some embodiments, a dark body is not counted unless: 1) its size inside the cell is greater than a minimum size for the anomaly expected, e.g., a minimum parasite size; 2) its aspect ratio (longest dimension divided by shorted dimension) is in a desired range; or 3) its pixel values have a desired distribution, or some combination of these conditions.

In some embodiments, step 370 includes storing the data for anomalies inside cells in the database that can be accessed by remote experts, in association with the corresponding digital image.

Step 379 represents a branch point in which it is determined whether another digital image from the same sample is to be included in the analysis. If so, control passes back to step 330 to process the next digital image as the current digital image. If not, control passes to step 380 to determine an analytical result for the sample.

In step 380, an analytical result is determined for the sample. In the illustrated embodiment, step 380 includes generating a parasitemia level for the sample, based on the counts of parasites inside red blood cells and the count of red blood cells. In some embodiments, step 380 involves not including a dark body in a count of parasites if it does not fit within size limits for a parasite. The size of the parasite can be determined from looking at two or more adjacent depths of focus. A dark body is not considered a parasite if its size is less than a minimum parasite size at all depths of focus or its size is greater than a maximum size when considering all depths of focus. This size analysis is performed for every dark body found inside a cell. In some embodiments, determining the parasitemia levels includes determining the number of parasites per volume of blood, by multiplying the number of parasites per red blood cell by the number of red blood cells per unit volume of blood. In some embodiments, determining the parasitemia levels includes determining the number of parasites per volume of blood, by determining the number of parasites per white blood cell and multiplying the number of parasites per white blood cell by the number of white blood cells per unit volume of blood.

In some embodiments, step 380 includes generating a parasitemia level based on a calibration factor determined in step 398, described below, for previous biological samples. For example, the calibration factor may increase or decrease the parasitemia level computed based on the count per red blood cell to account for the fact that the number of red blood cells per volume of blood are depressed in an infected individual. This calibration factor may be a function of a characteristic observable in the microscopic views, such as the average number of red blood cells per field of view or the average number of parasites per field of view, or the ratio of the two.

In the illustrated embodiment, step 380 also includes generating data that indicates the stage of malaria parasite detected in the cells and the number per stage. The stage of a parasite can be determined by the shape or shape distribution of the parasites inside the cells. In an example embodiment, the shape of the parasite is determined by the cross section of the same parasite in each field of view at several depths of focus. A parasite in one field of view at one depth of focus is considered the same parasite at a similar horizontal location in a second field of view at the next depth of focus, provided the next field of focus is changed in depth by less than the size of the parasite and the similar horizontal location is within the size of the parasite. Using this approach, it can be determined whether the parasite is spherical, elongated, ring-shaped, or banana-shaped, which shapes are characteristic of different stages of different strains of malaria. In some embodiments, step 380 includes providing a count of parasites per shape per red blood cell or volume of blood. The number of parasites per shape can also indicate the stage of the infestation. In the illustrated embodiment, step 380 also includes generating data that indicates the species (or strain) of malaria detected in the cells. The species is determined based on the distribution of the number of parasites of certain shapes in the thin smear sample.

In some embodiments, step 380 includes generating a stage or strain identification based on a calibration factor determined in step 398, described below, for previous biological samples. For example, the calibration factor may change the stage or strain identification based on the detected shapes to account for the fact that some shapes are easier to detect than another. This calibration factor may be a function of a characteristic observable in the microscopic views, such as the average number of parasites per size or shape category, or some combination of these.

In some embodiments, step 380 includes storing data that indicates some or all of the analytical results in the database that can be accessed by remote experts, in association with the corresponding digital images.

In step 398, a calibration factor is determined by comparing the automatically generated analytical result to some standard, such an extraordinarily careful analysis by an exceptional expert. In some embodiments, the calibration factor is a constant. As described above, in some embodiments, the calibration factor is a function of some observable property of the sample, such as the number of red blood cells per field of view, or number of parasites per field of view, or their ratio or some other factor. For example, the calibration factor in some embodiments is a function of the ratio of red blood cells to white blood cells.

In some embodiments, step 398 includes storing data that indicates the calibration factor and values of the observable upon which the calibration factor depends, if any, in the database that can be accessed by remote experts, in association with the corresponding digital images.

The method 300 described here is capable of detecting parasitemia levels in a rapid, quantifiable and repeatable manner that improves over current approaches for detecting and quantifying parasitemia levels in microscope images.

3. Hardware Overview

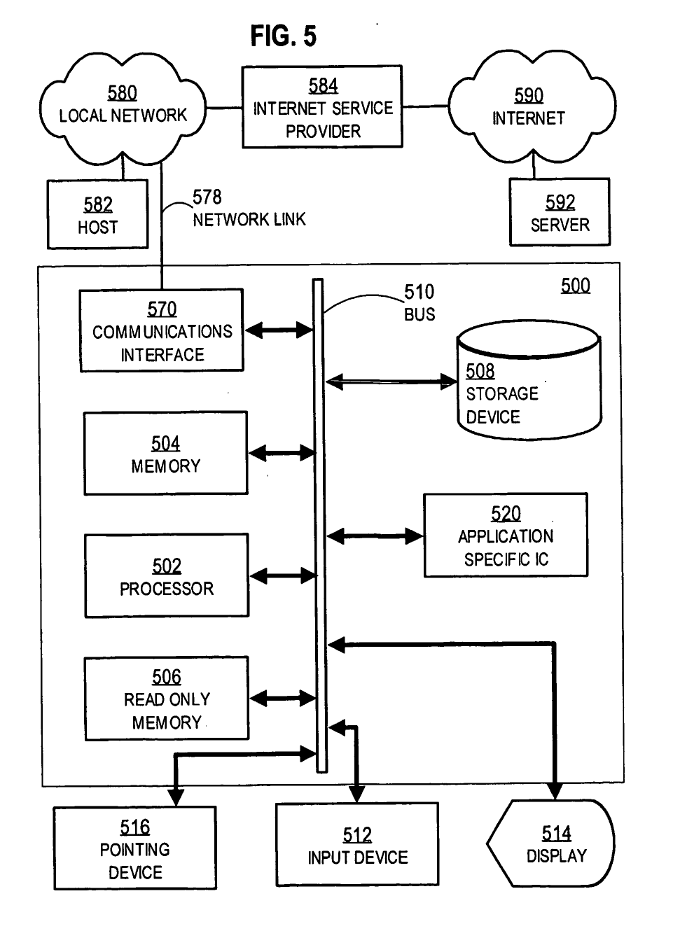

FIG. 5 is a block diagram that illustrates a computer system 500 upon which an embodiment of the invention may be implemented. Computer system 500 includes a communication mechanism such as a bus 510 for passing information between other internal and external components of the computer system 500. Information is represented as physical signals of a measurable phenomenon, typically electric voltages, but including, in other embodiments, such phenomena as magnetic, electromagnetic, pressure, chemical, molecular and atomic interactions. For example, north and south magnetic fields, or a zero and non-zero electric voltage, represent two states (0, 1) of a binary digit (bit). A sequence of binary digits constitutes digital data that is used to represent a number or code for a character. A bus 510 includes many parallel conductors of information so that information is transferred quickly among devices coupled to the bus 510. One or more processors 502 for processing information are coupled with the bus 510. A processor 502 performs a set of operations on information. The set of operations include bringing information in from the bus 510 and placing information on the bus 510. The set of operations also typically include comparing two or more units of information, shifting positions of units of information, and combining two or more units of information, such as by addition or multiplication. A sequence of operations to be executed by the processor 502 constitute computer instructions.

Computer system 500 also includes a memory 504 coupled to bus 510. The memory 504, such as a random access memory (RAM) or other dynamic storage device, stores information including computer instructions. Dynamic memory allows information stored therein to be changed by the computer system 500. RAM allows a unit of information stored at a location called a memory address to be stored and retrieved independently of information at neighboring addresses. The memory 504 is also used by the processor 502 to store temporary values during execution of computer instructions. The computer system 500 also includes a read only memory (ROM) 506 or other static storage device coupled to the bus 510 for storing static information, including instructions, that is not changed by the computer system 500. Also coupled to bus 510 is a non-volatile (persistent) storage device 508, such as a magnetic disk or optical disk, for storing information, including instructions, that persists even when the computer system 500 is turned off or otherwise loses power.

Information, including instructions, is provided to the bus 510 for use by the processor from an external input device 512, such as a keyboard containing alphanumeric keys operated by a human user, or a sensor. A sensor detects conditions in its vicinity and transforms those detections into signals compatible with the signals used to represent information in computer system 500. Other external devices coupled to bus 510, used primarily for interacting with humans, include a display device 514, such as a cathode ray tube (CRT) or a liquid crystal display (LCD), for presenting images, and a pointing device 516, such as a mouse or a trackball or cursor direction keys, for controlling a position of a small cursor image presented on the display 514 and issuing commands associated with graphical elements presented on the display 514.

In the illustrated embodiment, special purpose hardware, such as an application specific integrated circuit (IC) 520, is coupled to bus 510. The special purpose hardware is configured to perform operations not performed by processor 502 quickly enough for special purposes. Examples of application specific ICs include graphics accelerator cards for generating images for display 514, cryptographic boards for encrypting and decrypting messages sent over a network, speech recognition, and interfaces to special external devices, such as robotic arms and medical scanning equipment that repeatedly perform some complex sequence of operations that are more efficiently implemented in hardware.

Computer system 500 also includes one or more instances of a communications interface 570 coupled to bus 510. Communication interface 570 provides a two-way communication coupling to a variety of external devices that operate with their own processors, such as printers, scanners and external disks. In general the coupling is with a network link 578 that is connected to a local network 580 to which a variety of external devices with their own processors are connected. For example, communication interface 570 may be a parallel port or a serial port or a universal serial bus (USB) port on a personal computer. In some embodiments, communications interface 570 is an integrated services digital network (ISDN) card or a digital subscriber line (DSL) card or a telephone modem that provides an information communication connection to a corresponding type of telephone line. In some embodiments, a communication interface 570 is a cable modem that converts signals on bus 510 into signals for a communication connection over a coaxial cable or into optical signals for a communication connection over a fiber optic cable. As another example, communications interface 570 may be a local area network (LAN) card to provide a data communication connection to a compatible LAN, such as Ethernet. Wireless links may also be implemented. For wireless links, the communications interface 570 sends and receives electrical, acoustic or electromagnetic signals, including infrared and optical signals, that carry information streams, such as digital data. Such signals are examples of carrier waves.

The term computer-readable medium is used herein to refer to any medium that participates in providing instructions to processor 502 for execution. Such a medium may take many forms, including, but not limited to, non-volatile media,volatile media and transmission media. Non-volatile media include, for example, optical or magnetic disks, such as storage device 508. Volatile media include, for example, dynamic memory 504. Transmission media include, for example, coaxial cables, copper wire, fiber optic cables, and waves that travel through space without wires or cables, such as acoustic waves and electromagnetic waves, including radio, optical and infrared waves. Signals that are transmitted over transmission media are herein called carrier waves.

Common forms of computer-readable media include, for example, a floppy disk, a flexible disk, a hard disk, a magnetic tape, or any other magnetic medium, a compact disk ROM (CD-ROM), or any other optical medium, punch cards, paper tape, or any other physical medium with patterns of holes, a RAM, a programmable ROM (PROM), an erasable PROM (EPROM), a FLASH-EPROM, or any other memory chip or cartridge, a carrier wave, or any other medium from which a computer can read.

Network link 578 typically provides information communication through one or more networks to other devices that use or process the information. For example, network link 578 may provide a connection through local network 580 to a host computer 582 or to equipment 584 operated by an Internet Service Provider (ISP). ISP equipment 584 in turn provides data communication services through the public, world-wide packet-switching communication network of networks now commonly referred to as the Internet 590. A computer called a server 592 connected to the Internet provides a service in response to information received over the Internet. For example, server 592 provides information representing video data for presentation at display 514.

The invention is related to the use of computer system 500 for implementing the techniques described herein. According to one embodiment of the invention, those techniques are performed by computer system 500 in response to processor 502 executing one or more sequences of one or more instructions contained in memory 504. Such instructions, also called software and program code, may be read into memory 504 from another computer-readable medium such as storage device 508. Execution of the sequences of instructions contained in memory 504 causes processor 502 to perform the method steps described herein. In alternative embodiments, hardware, such as application specific integrated circuit 520, may be used in place of or in combination with software to implement the invention. Thus, embodiments of the invention are not limited to any specific combination of hardware and software.

The signals transmitted over network link 578 and other networks through communications interface 570, which carry information to and from computer system 500, are exemplary forms of carrier waves. Computer system 500 can send and receive information, including program code, through the networks 580, 590 among others, through network link 578 and communications interface 570. In an example using the Internet 590, a server 592 transmits program code for a particular application, requested by a message sent from computer 500, through Internet 590, ISP equipment 584, local network 580 and communications interface 570. The received code may be executed by processor 502 as it is received, or may be stored in storage device 508 or other non-volatile storage for later execution, or both. In this manner, computer system 500 may obtain application program code in the form of a carrier wave.

Various forms of computer readable media may be involved in carrying one or more sequence of instructions or data or both to processor 502 for execution. For example, instructions and data may initially be carried on a magnetic disk of a remote computer such as host 582. The remote computer loads the instructions and data into its dynamic memory and sends the instructions and data over a telephone line using a modem. A modem local to the computer system 500 receives the instructions and data on a telephone line and uses an infra-red transmitter to convert the instructions and data to an infra-red signal, a carrier wave serving as the network link 578. An infrared detector serving as communications interface 570 receives the instructions and data carried in the infrared signal and places information representing the instructions and data onto bus 510. Bus 510 carries the information to memory 504 from which processor 502 retrieves and executes the instructions using some of the data sent with the instructions. The instructions and data received in memory 504 may optionally be stored on storage device 508, either before or after execution by the processor 502.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A method for automatically analyzing a biological sample with a microscope, comprising the steps of:
obtaining a first digital image of a first field of view, in the microscope, of the biological sample;
automatically determining cell data that indicates an area co-located in the first digital image with a cell set of one or more cells of a particular type;
automatically determining anomalous data that indicates an area co-located in the first digital image with an anomalous set of zero or more particular objects that are anomalous to normal cells of the particular type;
automatically combining the cell data and the anomalous data to determine the particular objects inside the cell set of one or more cells of the particular type in the first digital image; and
generating an analytical result for the biological sample based on the particular objects inside the cell.

2. The method as recited in claim 1, said step of obtaining the first digital image further comprising the step of archiving the first digital image in a database that can be accessed by at least one of a local user and a remote user.

3. The method as recited in claim 1, said step of generating the analytical result further comprising the step of archiving the analytical result in association with the first digital image in a database that can be accessed by at least one or a local user and a remote user.

4. The method as recited in claim 1, said step of obtaining the first digital image further comprising obtaining the first digital image of a plurality of digital images corresponding to a plurality of fields of view of the microscope by performing the steps of:
automatically controlling a horizontal position of a platform holding the biological sample relative to the microscope to achieve a current horizontal position;
automatically controlling a depth of focus of the microscope to achieve a current depth of focus; and
automatically scanning a scan set of one or more picture elements of the first digital image for the current horizontal position and the current depth of focus.

5. The method as recited in claim 4, said step of automatically controlling the depth of focus further comprising the steps of:
holding the depth of focus constant for the first digital image; and
changing the depth of focus for a different, second digital image of the plurality of digital images by a depth increment based on a size associated with the particular objects anomalous to cells of the particular type.

6. The method as recited in claim 4,wherein;
said step of obtaining the first digital image comprises obtaining the plurality of digital images;
said step of automatically determining cell data in the first digital image comprises automatically determining cell data in each digital image of the plurality of digital images;
said step of automatically determining anomalous data in the first digital image comprises automatically determining anomalous data in each digital image of the plurality of digital images;
said step of automatically combining the cell data and the anomalous data to determine the particular objects inside the cell in the first digital image comprises automatically combining the cell data and the anomalous data in each digital image of the plurality of digital images to determine the particular objects inside the cell in each digital image of the plurality of digital images; and
said step of generating the analytical result further comprises determining a statistic for the biological sample based on a number of the particular objects inside the cell in every digital image of the plurality of digital images.

7. The method as recited in claim 6, wherein:
said step of obtaining the first digital image further comprises determining a target accuracy for quantifying the statistic for the sample; and
said step of automatically controlling the horizontal position further comprising the step of controlling the horizontal position to place in the plurality of digital images a percentage less than 100% of an area of the biological sample on the platform; and
the percentage is based on the target accuracy.

8. The method as recited in claim 1, said step of automatically combining the cell data and the anomalous data further comprising determining a number of the particular objects inside the cell set.

9. The method as recited in claim 8, said step of automatically determining cell data further comprising determining a number of cells in the cell set.

10. The method as recited in claim 9, said step of automatically combining the cell data and the anomalous data further comprising determining a level per cell based on the number of the particular objects inside the cell set and the number of cells in the cell set.

11. The method as recited in claim 10, said step of generating the analytical result further comprising determining a level per unit volume based on a normal number of the cells per unit volume.

12. The method as recited in claim 1, said step of generating the analytical result further comprising determining a stage of a disease based at least in part on the anomalous data associated with the particular objects inside the cell set.

13. The method as recited in claim 12, said step of generating the analytical result further comprising determining a number the particular objects inside the cell set associated with the stage of the disease.

14. The method as recited in claim 12, said step of determining the stage of the disease further comprising determining a shape of the particular objects inside the cell set.

15. The method as recited in claim 14, wherein:
said step of obtaining the first digital image further comprises obtaining a second digital image of a second field of view of the biological sample at a different depth of focus of the microscope;
said step of automatically determining anomalous data in the first digital image further comprises automatically determining anomalous data in the second digital image; and
said step of determining the shape of the particular objects inside the cell set further comprising determining the shape of a first particular object inside the cell set by performing the steps of
determining a location of the first particular object in the first digital image based at least in part on the anomalous data in the first digital image,
automatically determining second anomalous data in the anomalous data of the second digital image that is in a horizontal vicinity of the location of the first particular object in the first digital image, and
determining the shape of the first particular object based at least in part on the second anomalous data.

16. The method as recited in claim 1, said step of generating the analytical result further comprising determining a strain of a disease based at least in part on the anomalous data associated with the particular objects inside the cell set.

17. The method as recited in claim 1, said step of determining the cell data further comprising determining pixels above a threshold value in a color plane of the digital image.

18. The method as recited in claim 1, said step of determining the cell data further comprising determining pixels above a threshold value in a weighted sum of at least two color planes of the digital image.

19. The method as recited in claim 1, said step of determining the anomalous data further comprising determining pixels above a threshold value in a color plane of the digital image.

20. The method as recited in claim 1, said step of determining the anomalous data further comprising determining pixels above a threshold value in a weighted sum of at least two color planes of the digital image.

21. The method as recited in claim 17, said step of determining the anomalous data further comprising determining pixels above a different threshold value in a color plane of the digital image.

22. The method as recited in claim 18, said step of determining the anomalous data further comprising determining pixels above a different threshold value in the weighted sum of at least two color planes of the digital image.

23. The method as recited in claim 18, said step of determining the anomalous data further comprising determining pixels above a different threshold value in a different weighted sum of at least two color planes of the digital image.

24. The method as recited in claim 1, said step of determining the cell data further comprising determining edges in the digital image.

25. The method as recited in claim 1, said step of determining the anomalous data further comprising determining edges in the digital image.

26. The method as recited in claim 1, wherein:
the method further comprising determining a calibration factor for producing an analytical result that agrees with a standard; and
said step of generating the analytical result further comprises generating the analytical result based at least in part on the calibration factor.

27. The method as recited in claim 26, wherein:
said step of determining a calibration factor further comprising determining a calibration factor that depends on a parameter that can be derived from a digital image of a biological sample; and
said step of generating the analytical result further comprises determining the parameter based on the first digital image, and generating the calibration factor based on the parameter.

28. The method as recited in claim 1, wherein the particular objects are parasites.

29. The method as recited in claim 1, wherein the particular type of cells is a red blood cell.

30. The method as recited in claim 28, wherein the parasites are Malaria parasites.

31. The method as recited in claim 1, wherein the biological sample is a blood smear and the analytical result includes an indication of one of a presence of Malaria parasites and an absence of Malaria parasites.

32. The method as recited in claim 1, wherein the biological sample is a blood smear and the analytical result includes a parasitemia level for Malaria.

33. The method as recited in claim 1, wherein the biological sample is a blood smear and the analytical result includes an identification of Malaria parasite stage.

34. The method as recited in claim 1, wherein the biological sample is a blood smear and the analytical result includes an identification of Malaria parasite strain.

35. The method as recited in claim 1, wherein the particular objects are anomalously-shaped cell structures.

36. The method as recited in claim 35, wherein the analytical result includes an indication of one of a presence of babesiosis and an absence of babesiosis.

37. A computer-readable medium carrying one or more sequences of instructions for automatically analyzing a biological sample with a microscope, wherein execution of the one or more sequences of instructions by one or more processors causes the one or more processors to perform the steps of:
receiving a first digital image of a first field of view, in the microscope, of the biological sample;
determining cell data that indicates an area co-located in the first digital image with a cell set of one or more cells of a particular type;
determining anomalous data that indicates an area co-located in the first digital image with a anomalous set of zero or more particular objects that are anomalous to normal cells of the particular type;
combining the cell data and the anomalous data to determine the particular objects inside the cell set of one or more cells of the particular type in the first digital image; and
generating an analytical result for the biological sample based on the particular objects inside the cell.

38. A system for automatically analyzing a biological sample with a microscope, comprising:
a digital microscope for generating a first digital image of a first field of view of the biological sample;
a processor connected to the digital microscope; and
a computer readable medium storing one or more sequences of instructions which, when executed by the processor, cause the processor to carry out the steps of:
receiving the first digital image;
determining cell data that indicates an area co-located in the first digital image with a cell set of one or more cells of a particular type;
determining anomalous data that indicates an area co-located in the first digital image with a anomalous set of zero or more particular objects that are anomalous to normal cells of the particular type;
combining the cell data and the anomalous data to determine the particular objects inside the cell set of one or more cells of the particular type in the first digital image; and
generating an analytical result for the biological sample based on the particular objects inside the cell.

39. The system as recited in claim 38, wherein the analytical result is used to assist in a clinical diagnosis of a medical condition of a donor of the biological sample.

40. The system as recited in claim 38, wherein the analytical result provides a clinical diagnosis of a medical condition of a donor of the biological sample.
